(19) United States
(12) Patent Application Publication
(10) Pub. No.: US 2015/0105662 A1
(43) Pub. Date: Apr. 16, 2015

(54) DEVICE FIXING ADAPTOR AND ULTRASOUND PROBE SYSTEM

(71) Applicants: Kabushiki Kaisha Toshiba, Minato-ku (JP); Toshiba Medical Systems Corporation, Otawara-shi (JP)

(72) Inventors: Kentaro TSUZUKI, Nasushiobara (JP); Hiroyuki SHIKATA, Nasushiobara (JP); Takashi TAKEUCHI, Otawara (JP); Hideki KOSAKU, Nasushiobara (JP)

(73) Assignees: Kabushiki Kaisha Toshiba, Minato-ku (JP); Toshiba Medical Systems Corporation, Otawara-shi (JP)

(21) Appl. No.: 14/575,657

(22) Filed: Dec. 18, 2014

Related U.S. Application Data
(63) Continuation of application No. PCT/JP2013/070649, filed on Jul. 30, 2013.

(30) Foreign Application Priority Data
Jul. 30, 2012 (JP) 2012-168793
Jul. 30, 2013 (JP) 2013-158097

(51) Int. Cl. A61B 8/00; A61B 8/08

(52) U.S. Cl. CPC A61B 8/4455; A61B 8/0841; A61B 8/4477; A61B 8/546; A61B 8/4254
USPC 600/439; 600/459; 600/461

(57) ABSTRACT

A device fixing adaptor according to an embodiment fixes a device disposed at a grip part of an ultrasound probe to the ultrasound probe, by having such a shape that causes the device and a cable connected to the device to be pressed against the grip part from a top face thereof and inhibits the device from shifting a position thereof around the grip part.

### CROSS-REFERENCE TO RELATED APPLICATIONS

This application is a continuation of PCT international application Ser. No. PCT/JP2013/070649 filed on Jul. 30, 2013 which designates the United States, incorporated herein by reference, and which claims the benefit of priority from Japanese Patent Application No. 2012-168793, filed on Jul. 30, 2012, and Japanese Patent Application No. 2013-158097, filed on Jul. 30, 2013, the entire contents of which are incorporated herein by reference.

### FIELD

Embodiments described herein relate generally to a device fixing adaptor and an ultrasound probe system.

### BACKGROUND

Ultrasound diagnosis apparatuses receive reflected waves from the inside of an examined subject (hereinafter, a “subject”) as a result of scanning the inside of the subject with ultrasound waves and to further generate an ultrasound image obtained by imaging the state on the inside of the subject on the basis of reception signals generated from the reflected waves. More specifically, ultrasound diagnosis apparatuses transmit the ultrasound waves to the inside of the subject from an ultrasound probe and to generate the reception signals by receiving the reflected waves with the ultrasound probe, the reflected waves occurring due to mismatching of acoustic impedances on the inside of the subject.

Furthermore, to make it possible to perform an advanced diagnosis process by using an ultrasound diagnosis apparatus, various devices can be connected to an ultrasound probe. Such a device is held by an exclusive-use adaptor and connected to the ultrasound probe. Examples of such a device include a puncture needle. In that situation, a puncture adaptor that guides the puncture needle is attached to the ultrasound probe.

Furthermore, to make it possible to perform a more advanced diagnosis process by using an ultrasound diagnosis apparatus, a device having a cable therewith may be fixed to an ultrasound probe by using an exclusive-use adaptor designed for the device. The cable is connected to the device for the purpose of supplying electric power to the device or for the purpose of recording information obtained by the device into an external apparatus. Examples of such a device having a cable therewith include a position sensor used for detecting positions within a subject on whom an image taking process is performed. Besides the position sensor, other examples of such a device having a cable therewith include a light emitting device used for improving visibility of a diagnosed site, a treatment tool that expands the scope of a treatment method implemented by using the ultrasound probe, and a digital camera that obtains a photograph of a diagnosed site.

By connecting a device to the ultrasound probe in this manner, it is possible to expand the scope of a diagnosis process employing the ultrasound diagnosis apparatus, which is a relatively compact medical image diagnosis apparatus. It is therefore possible to obtain a larger amount of information at a lower cost.

When the connected device has a cable therewith, however, there are some situations where a conventional exclusive-use adaptor lowers the operability of the ultrasound probe.

### BRIEF DESCRIPTION OF THE DRAWINGS

FIG. 1 is a perspective view of an exterior of an ultrasound diagnosis apparatus to which a device fixing adaptor according to a first embodiment is applied;

FIG. 2, FIG. 3, FIG. 4 and FIG. 5 are drawings for explaining a conventional exclusive-use adaptor;

FIG. 6, FIG. 7, FIG. 8, FIG. 9A, FIG. 9B, FIG. 10A, FIG. 10B and FIG. 11 are drawings for explaining the device fixing adaptor according to the first embodiment;

FIG. 12 is a drawing for explaining a device fixing adaptor according to a second embodiment;

FIG. 13 is a drawing for explaining a device fixing adaptor according to a third embodiment; and

FIG. 14 is a drawing for explaining a device fixing adaptor according to a fourth embodiment.

### DETAILED DESCRIPTION

A device fixing adaptor according to an embodiment fixes a device disposed at a grip part of an ultrasound probe to the ultrasound probe, by having such a shape that causes the device and a cable connected to the device to be pressed against the grip part from a top face thereof and inhibits the device from shifting a position thereof around the grip part.

Exemplary embodiments of a device fixing adaptor will be explained in details, with reference to the accompanying drawings. The device fixing adaptor explained below is an adaptor used for fixing a device that has a cable therewith, to an ultrasound probe used in an image taking process performed by an ultrasound diagnosis apparatus.

### First Embodiment

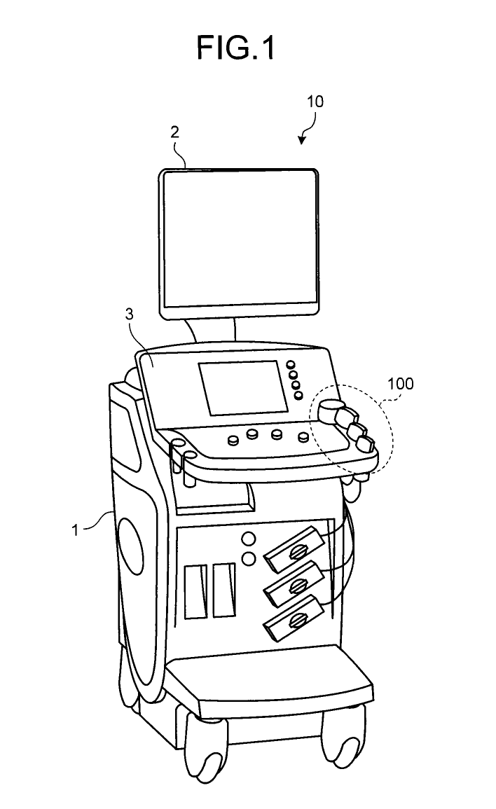

First, an overall configuration of an ultrasound diagnosis apparatus to which a device fixing adaptor according to a first embodiment is applied will be explained. FIG. 1 is a perspective view of an exterior of the ultrasound diagnosis apparatus to which the device fixing adaptor according to the first embodiment is applied. As illustrated in FIG. 1, an ultrasound diagnosis apparatus 10 with which the device fixing adaptor according to the first embodiment is used includes an apparatus main body 1, a monitor 2, and an operating unit 3. Furthermore, as illustrated in FIG. 1, the ultrasound diagnosis apparatus 10 is configured so that an ultrasound probe 100 of various kinds can detachably be connected to the apparatus main body 1 via a probe connector, in accordance with an image taking purpose or the like. Examples of the ultrasound probe 100 that can be connected to the apparatus main body 1 include a convex ultrasound probe, a sector ultrasound probe, a linear ultrasound probe, and a body-cavity ultrasound probe.

The apparatus main body 1 exercises overall control of the ultrasound diagnosis apparatus 10. For example, the apparatus main body 1 exercises various types of control related to the generation of an ultrasound image based on ultrasound reflected waves received by the ultrasound probe 100. The monitor 2 displays the ultrasound image generated by the apparatus main body 1 and the like and to display a Graphical User Interface (GUI) used by an operator of the ultrasound diagnosis apparatus 10 to input various types of setting requests through the operating unit 3. Furthermore, the operating unit 3 includes a trackball, a switch, a button, a touch command screen, and/or the like. The operating unit 3 receives the various types of setting requests from the operator of the ultrasound diagnosis apparatus 10 and to transfer the received various types of setting requests to the apparatus main body 1.

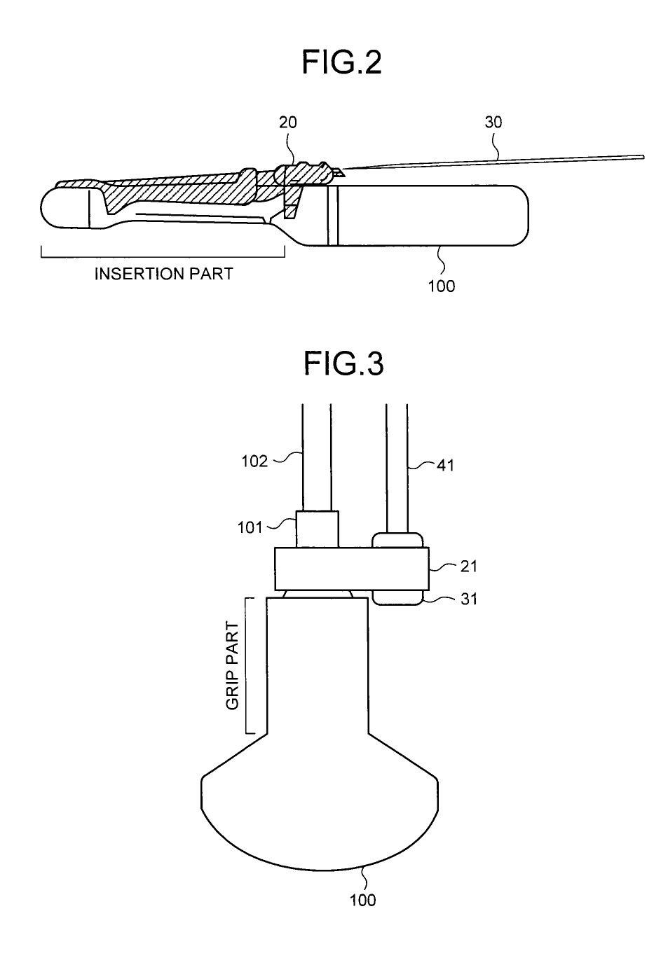

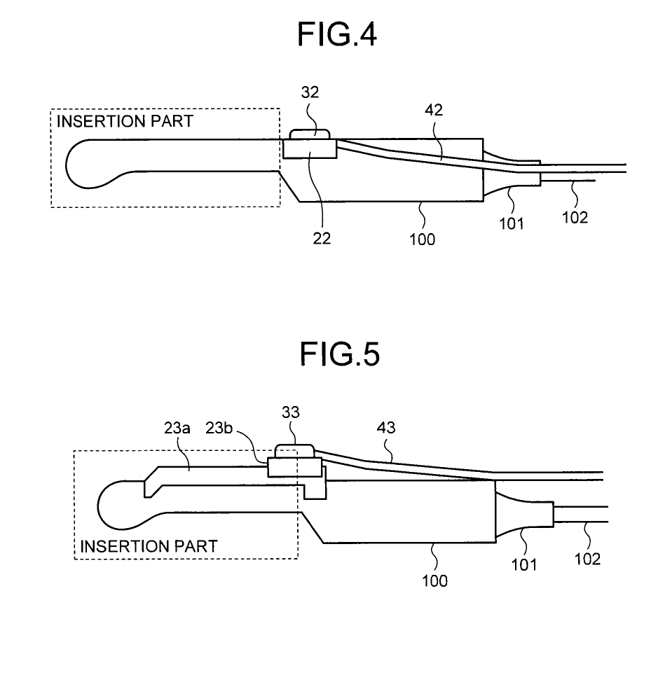

Conventionally, to make it possible to perform an advanced diagnosis process by using the ultrasound diagnosis apparatus 10, various devices can be connected to the ultrasound probe 100. Such a device is held by an exclusive-use adaptor and connected to the ultrasound probe. In the following sections, conventional exclusive-use adaptors will be explained with reference to FIGS. 2 to 5. FIGS. 2 to 5 are drawings for explaining the conventional exclusive-use adaptors.

Examples of such a device include a puncture needle used for performing a puncture process. The conventional exclusive-use adaptor illustrated in FIG. 2 is a puncture adaptor 20 that is connected to the ultrasound probe 100 when a puncture process is performed using a puncture needle 30 by employing the ultrasound probe 100, which is a body-cavity ultrasound probe. As illustrated in FIG. 2, the puncture adaptor 20 is connected to an insertion part of the ultrasound probe 100, the insertion part being inserted into the body of the subject. Although not shown in FIG. 2, a probe cover forming the insertion part of the ultrasound probe 100 is provided with a convex part where a portion of the insertion part is slightly protruded. The puncture adapter 20 is provided with a concave part interdigitating to the convex part of the insertion part. The concave part of the puncture adaptor 20 interdigitating to the convex part of the ultrasound probe 100, the puncture adaptor 20 is attached so as to have the insertion part sandwiched, as illustrated in FIG. 2. Furthermore, the puncture needle 30 is inserted into a guiding-purpose cylinder of the puncture adaptor 20.

The puncture adaptor 20 illustrated in FIG. 2 is shaped so as to fit the shape of the probe cover forming the insertion part of the ultrasound probe 100. With this arrangement, the operator (a medical doctor) is able to perform the puncture process without losing insertability of the ultrasound probe 100 into the subject's body or the operability of the ultrasound probe 100.

Furthermore, conventionally, to make it possible to perform a more advanced diagnosis process by using the ultrasound diagnosis apparatus 10, a device having a cable therewith may be fixed to the ultrasound probe. The cable is connected to the device for the purpose of supplying electric power to the device or for the purpose of recording information obtained by the device into an external apparatus. Examples of such a device having a cable therewith include a position sensor used for detecting positions within a subject on whom an image taking process is performed. Other examples of such a device having a cable therewith include a light emitting device used for improving visibility of a diagnosed site, a treatment tool that expands the scope of a treatment method implemented by using the ultrasound probe, and a digital camera that obtains a photograph of a diagnosed site.

Another conventional exclusive-use adaptor illustrated in FIG. 3 is a position sensor adaptor 21 used for fixing a position sensor 31 to the ultrasound probe 100, which is a convex ultrasound probe. As illustrated in FIG. 3, the position sensor 31 has, for example, a device cable 41 therewith for supplying electric power to the position sensor 31 and for transmitting information obtained by the position sensor 31 to the apparatus main body 1. A probe cable 102 illustrated in FIG. 3 is a cable for connecting the ultrasound probe 100 to the apparatus main body 1. For the purpose of preventing the probe cable 102 from bending at an end of a probe case, a cable bush 101 illustrated in FIG. 3 is wound around a part of the probe cable 102 disposed near a connection part connected to the ultrasound probe 100 and is configured by using, for example, rubber having elasticity.

As illustrated in FIG. 3, the position sensor adaptor 21 is fixed to the cable bush 101, whereas the position sensor 31 is fixed within the position sensor adaptor 21. For example, a cylindrical hollow is formed on the inside of the position sensor adaptor 21, as a fixing mechanism that fixes the position sensor 31. As illustrated in FIG. 3, because the position sensor 31 and the position sensor adaptor 21 are not disposed at the grip part of the ultrasound probe 100, the operator is able to handle the ultrasound probe 100 while there is no impact on the operability thereof.

Furthermore, a conventional exclusive-use adaptor illustrated in FIG. 4 is a position sensor adaptor 22 used for fixing a position sensor 32 having a device cable 42 therewith, to the ultrasound probe 100, which is a body-cavity ultrasound probe.

As illustrated in FIG. 4, the position sensor adaptor 22 is fixed to a part of a non-insertion part of the ultrasound probe 100 that is disposed closest to an insertion part, whereas the position sensor 32 is fixed onto the position sensor adaptor 22.

A conventional exclusive-use adaptor illustrated in FIG. 5 is a position sensor adaptor 23b used for fixing a position sensor 33 having a device cable 43 therewith, to the ultrasound probe 100, which is a body-cavity ultrasound probe. In this situation, the position sensor adaptor 23b illustrated in FIG. 5 is attached to a fixing mechanism provided on a puncture adaptor 23a that is exclusively used for a body-cavity ultrasound probe. In other words, the puncture adaptor 23a exclusively used for a body-cavity ultrasound probe and the position sensor adaptor 23b illustrated in FIG. 5 structure a function-combined adaptor for a puncture needle and the position sensor 33.

The ultrasound diagnosis apparatus 10 identifies the position of a cross-sectional plane of a taken ultrasound image, on the basis of position information obtained by any of the position sensors 31 to 33 that are fixed as illustrated in FIGS. 2 to 5. Furthermore, by using the position information, the operator is able to view, at the same time, the ultrasound image taken by the ultrasound diagnosis apparatus 10 and a medical image having the same cross-sectional plane as the ultrasound image, the medical image being taken by a medical image diagnosis apparatus other than the ultrasound diagnosis apparatus 10. As a result, it is possible to significantly increase the amount of information required by a diagnosis process.

The position sensor adaptor 21 illustrated in FIG. 3, however, fixes the position sensor 31 to a position that is relatively distant from an abutting plane where the ultrasound probe 100 and the body surface of the subject abut against each other. As a result, the position sensor 31 fixed to the position sensor adaptor 21 is not able to achieve a maximum level of precision in the position detection. Furthermore, if the position sensor adaptor 21 illustrated in FIG. 3 is an adaptor used for fixing a light emitting device such as a Light Emitting Diode (LED), because the light emitting device is fixed to the position distant from the abutting plane, the luminosity of the light radiated on the body surface of the subject is insufficient.

The reason is that the position sensor adaptor 21 is fixed to the cable bush 101, so that the device and the cable of the device are not disposed at the grip part of the ultrasound probe 100, for the purpose of maintaining the operability of the ultrasound probe 100.

In contrast, the conventional exclusive-use adaptors illustrated in FIGS. 4 and 5 (i.e., the position sensor adaptor 22 and the position sensor adaptor 23b) are able to minimize the distance between the body surface of the subject and the device. However, when the conventional exclusive-use adaptors illustrated in FIGS. 4 and 5 are used, because the cable of the device is disposed at the grip part of the ultrasound probe 100, the operability of the ultrasound probe 100 is significantly degraded. In particular, in the example of the position sensor adaptor 23b illustrated in FIG. 5, the puncture adaptor 23a exclusively used for a body-cavity ultrasound probe needs to be connected to the ultrasound probe 100, even if no puncture process is performed. For this reason, in order to obtain position information, the puncture adaptor 23a exclusively used for a body-cavity ultrasound probe, the position sensor adaptor 23b, and the position sensor 33 need to be attached to the ultrasound probe 100, which makes the exterior outline of the ultrasound probe 100 large. Thus, the operability of the ultrasound probe 100 is degraded.

Furthermore, as for the puncture adaptor 20 illustrated in FIG. 2 also, if a position sensor is attached to a tip end of the puncture needle 30 or if the puncture adaptor 20 is provided with a controlling mechanism that is able to adjust the puncture angle, the operability of the ultrasound probe 100 is degraded, because a cable is required.

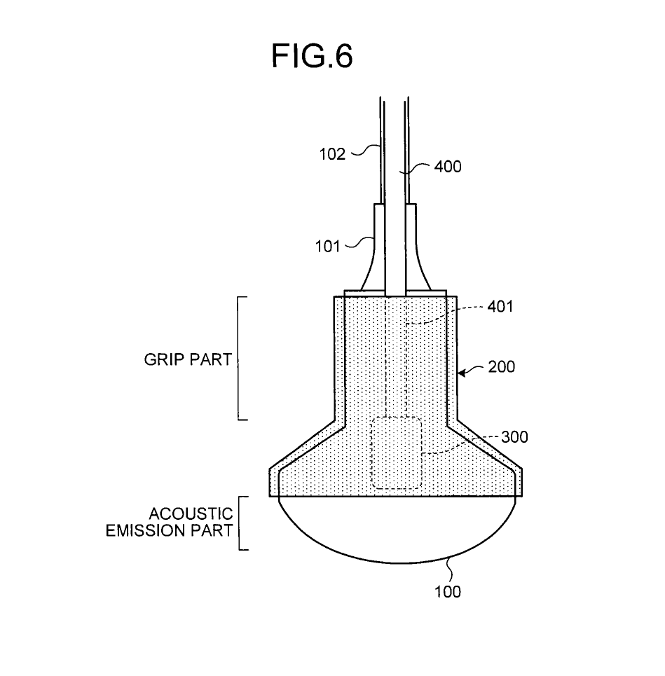

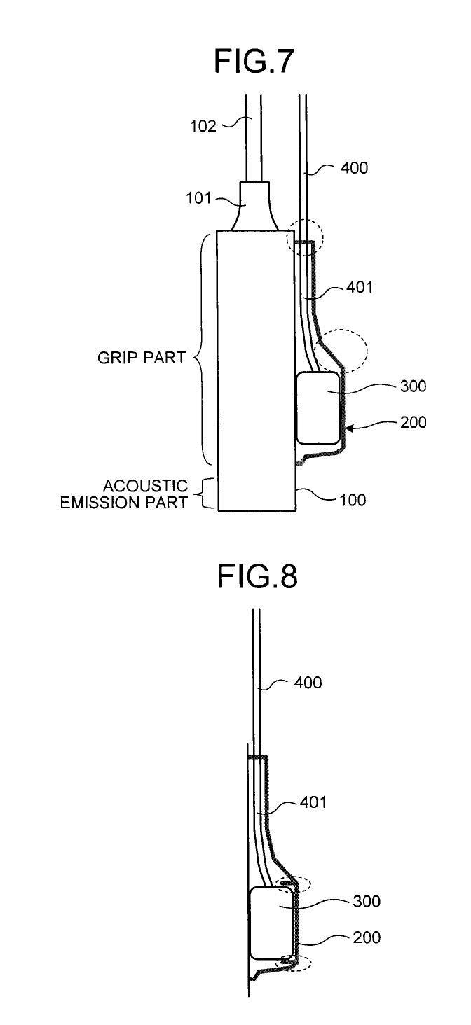

To cope with these situations, in a first embodiment, a device fixing adaptor described below is used, for the purpose of preventing the operability of the ultrasound probe 100 from being degraded even in the situation where the device fixed to the ultrasound probe 100 has a cable therewith. In other words, the device fixing adaptor described below has such a shape that causes a device disposed at the grip part of the ultrasound probe 100 and the cable connected to the device to be pressed against the grip part from a top face thereof and that inhibits the device from shifting the position thereof around the grip part. With this arrangement, the device fixing adaptor fixes the device to the ultrasound probe 100. More specifically, the device fixing adaptor according to the first embodiment encloses therein the device and a grip-part cable, which is a part of the cable disposed at the grip part of the ultrasound probe 100. In the following sections, an example of the device fixing adaptor according to the first embodiment will be explained, with reference to FIGS. 6 to 11. FIGS. 6 to 11 are drawings for explaining the device fixing adaptor according to the first embodiment.

In the first embodiment, an example will be explained in which, as illustrated in FIG. 6, a device fixing adaptor 200 fixes a position sensor 300 having a device cable 400 therewith, to the ultrasound probe 100, which is a convex ultrasound probe.

As illustrated in FIG. 6, the device fixing adaptor 200 according to the first embodiment fixes the position sensor 300 to the ultrasound probe 100, by enclosing therein the position sensor 300 connected to the device cable 400 and a grip-part cable 401 that is a part of the device cable 400 disposed at the grip part of the ultrasound probe 100. FIG. 6 illustrates a shape of the ultrasound probe 100 as viewed from above.

More specifically, the region enclosed in the device fixing adaptor 200 is a region extending from a position on the acoustic transmission part side of the position sensor 300, to a position where the grip-part cable 401 is provided. To enclose the grip-part cable 401 therein, the device fixing adaptor 200 is attached to the grip part of the ultrasound probe 100, as illustrated in FIG. 6, instead of being attached to the cable bush 101 that protects the probe cable 102. The device fixing adaptor 200 according to the first embodiment fixes the position sensor 300 and the grip-part cable 401 to the grip part of the ultrasound probe 100, by enclosing therein the position sensor 300 and the grip-part cable 401.

With this arrangement, according to the first embodiment, even when position information is obtained by the position sensor 300 having the device cable 400 therewith, it is possible to prevent the operability of the ultrasound probe 100 from being degraded.

In this situation, as illustrated in FIG. 6, for the purpose of maintaining the operability and the ease of grip of the ultrasound probe 100, the device fixing adaptor 200 is shaped so as to maintain the exterior outline of a cover forming the grip part of the ultrasound probe 100. Furthermore, to maintain the operability and the ease of grip of the ultrasound probe 100 as much as possible, it is desirable to configure the casing thickness of the device fixing adaptor 200 to be as thin as possible while ensuring the strength thereof.

Furthermore, in the first embodiment, as illustrated in FIG. 6, because the device fixing adaptor 200 is attached to the grip part of the ultrasound probe 100, the device fixing adaptor 200 is able to, at the grip part of the ultrasound probe 100, fix the position sensor 300 in such a manner that the position sensor 300 is disposed close to the acoustic emission part of the ultrasound probe 100.

In this situation, the fixation of the position sensor 300 by the device fixing adaptor 200 can be realized by either of the following two methods (i.e., a first fixing method and a second fixing method).

According to the first fixing method, as illustrated in FIG. 7, the device fixing adaptor 200 fixes the position sensor 300 by sandwiching the position sensor 300 between the device fixing adaptor 200 and the ultrasound probe 100. FIG. 7 is a cross-sectional view of the ultrasound probe 100 illustrated in FIG. 6. According to the first fixing method, as illustrated in FIG. 7, the device fixing adaptor 200 presses the position sensor 300 directly against the grip part of the ultrasound probe 100. As a result, according to the first method, it is possible to keep the size of the device fixing adaptor 200 as small as possible. In this situation, as mentioned above, the device fixing adaptor 200 is shaped so as to maintain the exterior outline of the cover forming the grip part of the ultrasound probe 100. In addition, to maintain the operability and the ease of grip of the ultrasound probe 100, the device fixing adaptor 200 is shaped so as to keep the difference in level from the ultrasound probe 100 small (see the dotted-line circle in FIG. 7).

According to the first fixing method, to prevent the position sensor 300 from moving within the space formed between the device fixing adaptor 200 and the grip part of the ultrasound probe 100 without fail, the device fixing adaptor 200 may further be provided with a fixing mechanism that fixes the position sensor 300, as exemplified in FIG. 8. In one example shown in FIG. 8, the device fixing adaptor 200 has protrusions for fixing the position sensor 300 (see the dotted-line oval in FIG. 8).

Furthermore, to fix the device fixing adaptor 200 to the grip part of the ultrasound probe 100, the device fixing adaptor 200 may be shaped so as to fit the shape of the outer circumference of the grip part of the ultrasound probe 100, as illustrated in FIG. 9A, for example. The device fixing adaptor 200 illustrated in FIG. 9A is shaped so as to enclose therein a probe cover forming the grip part of the ultrasound probe 100.

Alternatively, to fix the device fixing adaptor 200 to the grip part of the ultrasound probe 100, the device fixing adaptor 200 may further have an interdigitation mechanism that is interdigitated to an interdigitation mechanism of the ultrasound probe 100. In a case exemplified in FIG. 9B, one convex part each is formed on each of the two lateral faces of the probe cover forming the grip part of the ultrasound probe 100 so that two convex parts are formed. Furthermore, in the example shown in FIG. 9B, the device fixing adaptor 200 has formed therein concave parts that are interdigitated to the two convex parts of the probe cover.

In contrast, according to the second fixing method, as illustrated in FIG. 10A, the device fixing adaptor 200 has a wall 201 that forms a housing space for housing therein the position sensor 300 and the grip-part cable 401. In other words, according to the second fixing method, as illustrated in FIG. 10A, the device fixing adaptor 200 has the wall 201 that is shaped so as to fit the outer circumferences of the position sensor 300 and the grip-part cable 401.

Furthermore, according to the second fixing method, as illustrated in FIG. 10B, the device fixing adaptor 200 has, on a surface of the wall 201 that is in contact with the ultrasound probe 100, a fixing mechanism that fixes at least the position sensor 300 and the grip-part cable 401. In one example illustrated in FIG. 10B, a device holder 202 is provided on the surface of the wall 201 that is in contact with the ultrasound probe 100, the device holder 202 fixing the position sensor 300 to the device fixing adaptor 200. Furthermore, in the example illustrated in FIG. 10B, a cable holder 203 is provided on the surface of the wall 201 that is in contact with the ultrasound probe 100, the cable holder 203 fixing the grip-part cable 401 to the device fixing adaptor 200. In the second fixing method, it is also acceptable to provide a holder that entirely covers the surface of the wall 201 that is in contact with the ultrasound probe 100.

Furthermore, according to the second fixing method, as illustrated in FIG. 11, the device fixing adaptor 200 is configured so that the position sensor 300 fixed in the housing space formed by the wall 201 by the device holder 202 and the grip-part cable 401 fixed in the housing space formed by the wall 201 by the cable holder 203 are fixed to the ultrasound probe 100. According to the second fixing method, as explained with reference to FIG. 9A, the device fixing adaptor 200 is fixed to the ultrasound probe 100 by being shaped so as to fit the outer circumference of the grip part of the ultrasound probe 100. Alternatively, according to the second fixing method, as explained with reference to FIG. 9B, the device fixing adaptor 200 is fixed to the ultrasound probe 100 by having the interdigitation mechanism interdigitated to the interdigitation mechanism of the grip part of the ultrasound probe 100.

In this situation, as illustrated in FIG. 11, due to the thickness-direction dimensions of the device holder 202 and the cable holder 203, the thickness-direction dimension of the device fixing adaptor 200 according to the second fixing method is larger than the thickness-direction dimension of the device fixing adaptor 200 according to the first fixing method illustrated in FIG. 7.

As explained above, according to the first embodiment, the device fixing adaptor 200 has such a shape that causes the position sensor 300 disposed at the grip part of the ultrasound probe 100 and the cable 400 connected to the position sensor 300 to be pressed against the grip part from the top face thereof and that inhibits the position sensor 300 from shifting the position thereof around the grip part. With this arrangement, the device fixing adaptor 200 fixes the position sensor 300 to the ultrasound probe 100. More specifically, according to the first embodiment, the device fixing adaptor 200 is shaped so as to fix a main body of the position sensor 300 and the grip-part cable 401 to the ultrasound probe 100, by having the main body of the position sensor 300 and the grip-part cable 401 completely enclosed therein. Even more specifically, according to the first embodiment, the device fixing adaptor 200 is shaped so as to be able to completely enclose therein and fix the main body of the position sensor 300 and the grip-part cable 401, while the length thereof extends from a tip end of the grip part of the ultrasound probe 100 to the vicinity of the cable bush 101 of the ultrasound probe 100. Furthermore, according to the first embodiment, the exterior outline of the device fixing adaptor 200 is smoothly joined with the grip part of the ultrasound probe 100 and is shaped so as to maintain the shape of the ultrasound probe 100 as much as possible. With these arrangements, according to the first embodiment, it is possible to prevent the operability of the ultrasound probe 100 from being degraded.

Furthermore, according to the first embodiment, at the grip part of the ultrasound probe 100, the device fixing adaptor 200 fixes the position sensor 300 in such a manner that the position sensor 300 is disposed close to the acoustic emission part of the ultrasound probe 100. With this arrangement, according to the first embodiment, it is possible to utilize the capability of the position sensor 300 at the maximum, in comparison to the example of the position sensor adaptor 21 illustrated in FIG. 3.

Furthermore, according to the first embodiment, the device fixing adaptor 200 fixes the position sensor 300 and the grip-part cable 401 to the grip part of the ultrasound probe 100 by using either the first fixing method or the second fixing method. According to the first fixing method by which the position sensor 300 and the grip-part cable 401 are sandwiched between the ultrasound probe 100 and the device fixing adaptor 200, it is possible to keep the thickness-direction dimension of the device fixing adaptor 200 as small as possible.

In contrast, according to the second fixing method by which the holders are used, although the thickness-direction dimension of the device fixing adaptor 200 is slightly larger than the example using the first fixing method, it is possible to enhance the level of performance in fixing the position sensor 300 and the grip-part cable 401. According to the first fixing method, the grip-part cable 401 is fixed so as to be able to move, with freedom to some extent, within the space formed between the device fixing adaptor 200 and the grip part, insofar as the operability of the ultrasound probe 100 is not hindered. In other words, according to the first fixing method, the fixation is realized in such a manner that the grip-part cable 401 is inhibited from shifting the position thereof in the direction away from the grip part, but is given freedom to some extent, to move in the directions along the surface of the grip part. In contrast, according to the second fixing method, the grip-part cable 401 is inhibited from shifting the position thereof both in the direction away from the grip part and in the directions along the surface of the grip part.

Furthermore, according to the first embodiment, the device fixing adaptor 200 is fixed to the ultrasound probe 100 by being shaped so as to fit the shape of the outer circumference of the ultrasound probe 100. Alternatively, according to the first embodiment, the device fixing adaptor 200 is fixed to the ultrasound probe 100, by providing each of the ultrasound probe 100 and the device fixing adaptor 200 with interdigitation mechanism. In the former example, there is no need to perform a special processing process on the ultrasound probe 100; however, in the former example, if the ultrasound probe 100 had a point-symmetric shape (e.g., a perfect circle), the device fixing adaptor 200 would have a possibility of rotating. Thus, the former example is based on the premise that the ultrasound probe 100 has an asymmetrical shape. In contrast, as for the latter example, although it is necessary to perform a special processing process on the ultrasound probe 100, the latter example is applicable regardless to the shape of the ultrasound probe 100.

### Second Embodiment

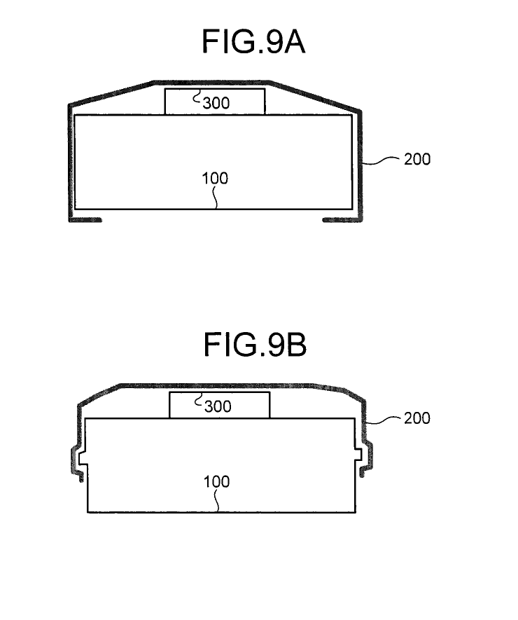

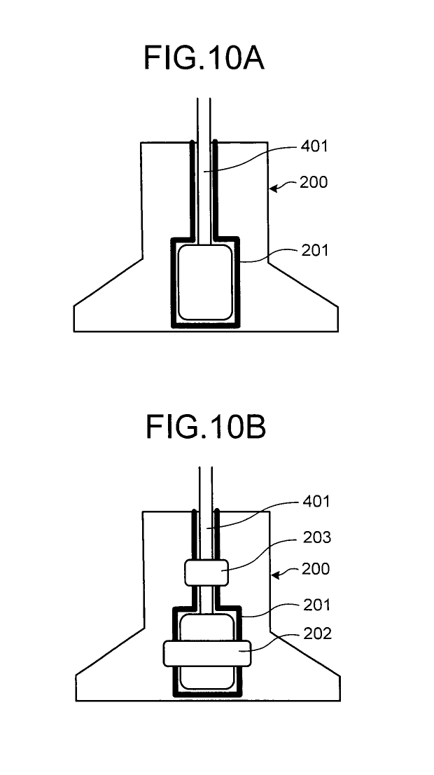

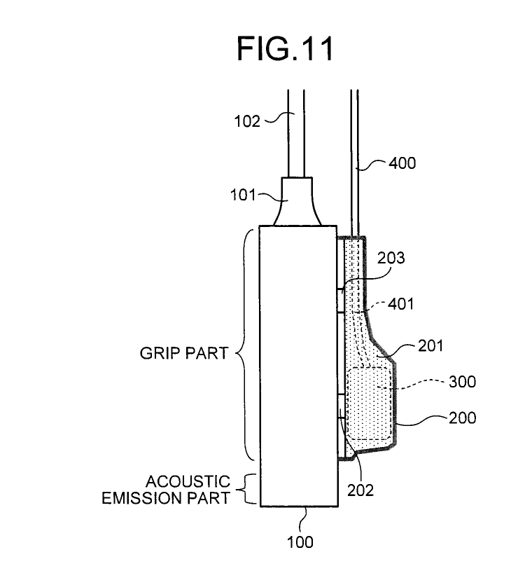

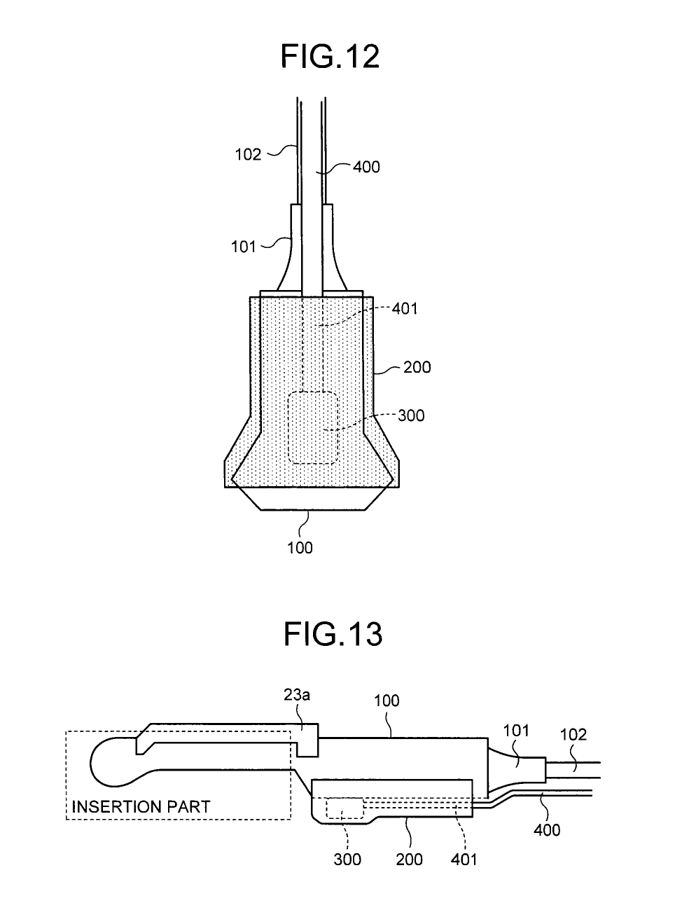

In a second embodiment, an example in which the ultrasound probe 100 is a sector ultrasound probe will be explained, with reference to FIG. 12. FIG. 12 is a drawing for explaining a device fixing adaptor according to the second embodiment.

As illustrated in FIG. 12, the device fixing adaptor 200 according to the second embodiment fixes the position sensor 300 to the sector ultrasound probe (i.e., the ultrasound probe 100), by enclosing therein the position sensor 300 having the device cable 400 therewith and the grip-part cable 401.

Similarly to the first embodiment, in the second embodiment also, the device fixing adaptor 200 that is shaped so as to fit the shape of the ultrasound probe 100 encloses therein the main body of the position sensor 300 and the grip-part cable 401. With this arrangement, in the second embodiment also, it is possible to fix the position sensor 300 in a position as close as possible to the body surface of the subject, without losing the operability or the ease of grip of the ultrasound probe 100. The configurations described in the first embodiment are simply applied to the device fixing adaptor 200 according to the second embodiment, except that the ultrasound probe 100 is a sector ultrasound probe.

Furthermore, in an example where a linear ultrasound probe is the ultrasound probe 100, it is possible to achieve the same or similar advantageous effects by shaping the device fixing adaptor 200 so as to fit the shape of the linear ultrasound probe.

### Third Embodiment

In a third embodiment, an example in which the ultrasound probe 100 is a body-cavity ultrasound probe will be explained, with reference to FIG. 13. FIG. 13 is a drawing for explaining a device fixing adaptor according to the third embodiment.

As illustrated in FIG. 13, the device fixing adaptor 200 according to the third embodiment fixes the position sensor 300 having the device cable 400 therewith to the body-cavity ultrasound probe (i.e., the ultrasound probe 100) by enclosing therein the position sensor 300 and the grip-part cable 401.

Similarly to the first and the second embodiments, in the third embodiment also, the device fixing adaptor 200 that is shaped so as to fit the shape of the ultrasound probe 100 encloses therein the main body of the position sensor 300 and the grip-part cable 401. With this arrangement, in the third embodiment also, it is possible to fix the position sensor 300 in a position as close as possible to the body surface of the subject, without losing the operability or the ease of grip of the ultrasound probe 100.

The configurations described in the first embodiment are simply applied to the device fixing adaptor 200 according to the third embodiment, except that the ultrasound probe 100 is a body-cavity ultrasound probe. It should be noted however that, because the body-cavity ultrasound probe has a shape approximating a perfect circle, it is desirable to apply the interdigitation mechanism described with reference to FIG. 9B to the fixation between the device fixing adaptor 200 and the ultrasound probe 100.

Furthermore, in the third embodiment, because the device fixing adaptor 200 that encloses therein the main body of the position sensor 300 and the grip-part cable 401 is used, the position sensor 300 is fixed to a portion of the non-insertion part that is disposed closest to the body surface of the subject. As a result, according to the third embodiment, it is possible to use the position sensor 300 and a puncture needle together. FIG. 13 illustrates an example in which the puncture adaptor 23a exclusively used for a body-cavity ultrasound probe illustrated in FIG. 5 and the device fixing adaptor 200 are attached to the body-cavity ultrasound probe. To not hinder the insertion of a puncture into the puncture adaptor 23a that is exclusively used for a body-cavity ultrasound probe, it is desirable to attach, as illustrated in FIG. 13, the device fixing adaptor 200 in a position on the plane opposite to the plane on which the puncture adaptor 23a exclusively used for a body-cavity ultrasound probe is provided.

Furthermore, generally speaking, body-cavity ultrasound probes are configured so that a probe cover has formed therewith a slip-off preventing mechanism such as a protrusion, a step, a groove, or the like that prevents a sheath enclosing the insertion part therein from slipping off. For this reason, it is desirable to shape the device fixing adaptor 200 according to the third embodiment so as to have a shape that reproduces such a slip-off preventing mechanism.

### Fourth Embodiment

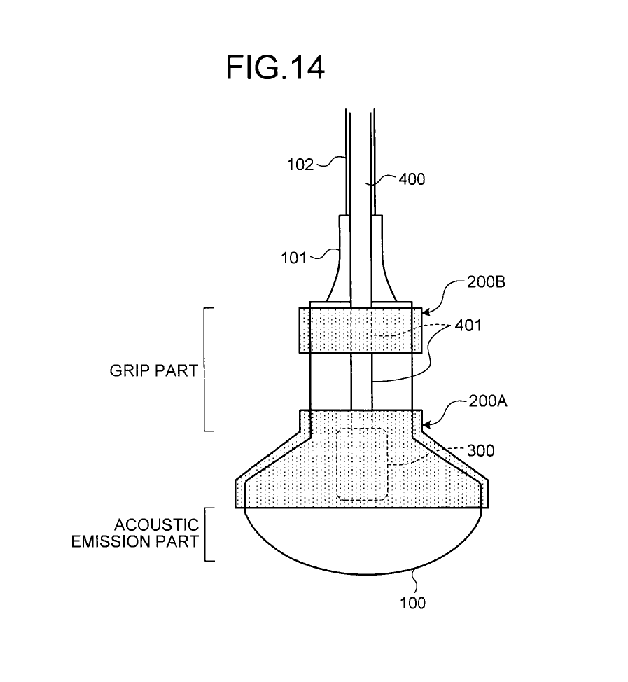

In the first to the third embodiments described above, the examples are explained in which the device is inhibited from shifting the position thereof around the grip part, by enclosing both the device and the grip-part cable. In a fourth embodiment, an example will be explained in which the device is inhibited from shifting the position thereof around the grip part, by enclosing the device and the grip-part cable independently of each other. FIG. 14 is a drawing for explaining the fourth embodiment.

FIG. 14 illustrates a device fixing adaptor 200A and a device fixing adaptor 200B that fixes the position sensor 300 connected to the device cable 400 to the ultrasound probe 100, which is a convex ultrasound probe. The device fixing adaptor 200 described in the first embodiment and illustrated in FIG. 6 inhibits the position sensor 300 from shifting the position thereof and to inhibit the grip-part cable 401 from shifting the position thereof, by enclosing therein both the position sensor 300 and the grip-part cable 401.

In contrast, according to the fourth embodiment, as illustrated in FIG. 14, the device fixing adaptor 200A inhibits the position sensor 300 from shifting the position thereof around the grip part, by enclosing therein the position sensor 300 while pressing the position sensor 300 against the grip part from the top face thereof. Furthermore, in the fourth embodiment, as illustrated in FIG. 14, the device fixing adaptor 200B inhibits the grip-part cable 401 from shifting the position thereof around the grip part, by enclosing therein a part of the grip-part cable 401 while pressing the part of the grip-part cable 401 against the grip part from the top face thereof. By using this configuration in which the position sensor 300 and the grip-part cable 401 are pressed down against the grip part independently of each other, it is also possible to prevent the operability of the ultrasound probe 100 from being degraded, similarly to the first to the third embodiments.

The device fixing adaptor 200A may fix the position sensor 300 by using either the first fixing method or the second fixing method described above. Furthermore, the device fixing adaptor 200B may fix the part of the grip-part cable 401 by using either the first fixing method or the second fixing method described above. Additionally, the configurations described in any of the first to the third embodiments are simply applied to the device fixing adaptor 200A and the device fixing adaptor 200B according to the fourth embodiment, except that the position sensor 300 and the grip-part cable 401 are enclosed independently of each other.

The first to the fourth embodiments are explained above using the examples in which the device having a cable therewith is the position sensor 300. The device fixing adaptors (200 or 200A and 200B) described in the first to the fourth embodiments above are also applicable to a situation where the device having a cable therewith is an acceleration sensor that is able to have the ultrasound probe 100 function as an input device or a vibration generating device that applies and releases pressure to and from the body surface of a subject for elastography purposes.

Furthermore, the device fixing adaptors (200 or 200A and 200B) described in the first to the fourth embodiments above are also applicable to a situation where the device having a cable therewith is a cooling device that circulates a cooling fluid to cool the ultrasound probe 100, a light emitting device that improves visibility of an image taking site, a radiation applying device that applies radiation such as X-rays, or an ultrasound gel supplying device that supplies ultrasound gel to be applied to the body surface of a subject so that ultrasound waves are transmitted and received efficiently.

Furthermore, the device fixing adaptors (200 or 200A and 200B) described in the first to the fourth embodiments above are also applicable to a situation where the device having a cable therewith is another ultrasound probe that is different from the ultrasound probe 100 and is used for realizing a bi-plane display.

Furthermore, the device fixing adaptors (200 or 200A and 200B) described in the first to the fourth embodiments above are also applicable to a situation where the device having a cable therewith is an optical image taking device such as a digital camera, a treatment device for performing radiofrequency ablation (RFA) or the like, or a puncture device with a puncture needle or the like to which a position sensor is attached to a tip end thereof.

As explained above, according to at least one aspect of the first to the fourth embodiments, it is possible to prevent the operability of the ultrasound probe from being degraded even when the device fixed to the ultrasound probe has a cable therewith.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A device fixing adaptor that fixes a device disposed at a grip part of an ultrasound probe to the ultrasound probe, by having such a shape that causes the device and a cable connected to the device to be pressed against the grip part from a top face thereof and that inhibits the device from shifting a position thereof around the grip part.

2. The device fixing adaptor according to claim 1, wherein the device fixing adaptor encloses therein the device and a grip-part cable, which is a part of the cable disposed at the grip part of the ultrasound probe.

3. The device fixing adaptor according to claim 1, wherein the device fixing adaptor encloses therein the device and a grip-part cable independently of each other, the grip-part cable being a part of the cable disposed at the grip part of the ultrasound probe.

4. The device fixing adaptor according to claim 1, wherein the device fixing adaptor is shaped so as to maintain an exterior outline of a cover forming the grip part of the ultrasound probe.

5. The device fixing adaptor according to claim 1, wherein the device fixing adaptor fixes the device by sandwiching the device between the device fixing adaptor and the ultrasound probe.

6. The device fixing adaptor according to claim 1, further comprising:
a wall that forms a housing space for housing therein the device and a grip-part cable, which is a part of the cable disposed at the grip part of the ultrasound probe; and
a fixing mechanism that is provided on a surface of the wall being in contact with the ultrasound probe and fixes at least the device and the grip-part cable.

7. The device fixing adaptor according to claim 1, further comprising an interdigitation mechanism that is interdigitated to an interdigitation mechanism of the ultrasound probe.

8. The device fixing adaptor according to claim 1, wherein, at the grip part of the ultrasound probe, the device fixing adaptor fixes the device in such a manner that the device is disposed close to an acoustic emission part of the ultrasound probe.

9. The device fixing adaptor according to claim 1, wherein the device is one of the following: a position sensor, an acceleration sensor, a vibration generating device, a cooling device, a light emitting device, a radiation applying device, an ultrasound gel supplying device, another ultrasound probe that is different from the ultrasound probe, an optical image taking device, a treatment device, and a puncture device.

10. An ultrasound probe system comprising:
an ultrasound probe that transmits and receive ultrasound waves;
a device disposed at a grip part of the ultrasound probe; and
a device fixing adaptor that fixes the device to the ultrasound probe by having such a shape that causes the device and a cable connected to the device to be pressed against the grip part from a top face thereof and that inhibits the device from shifting a position thereof around the grip part.
